(19) United States
(12) Patent Application Publication
(10) Pub. No.: US 2009/0214650 A1
(43) Pub. Date: Aug. 27, 2009

(54) Methods of Treating alcoholism and alcohol related disorders using combination drug therapy and swellable polymers

(75) Inventor: Elliot Ehrich, Lincoln, MA (US)

Correspondence Address: ELMORE PATENT LAW GROUP, PC, 515 Groton Road, Unit 1R, Westford, MA, 01886, US

(73) Assignee: Alkermes, Inc., Cambridge, MA (US)

(21) Appl. No.: 12/162,801

(22) PCT Filed: Feb. 1, 2007
(86) PCT No.: PCT/US2007/002972

§ 371 Date: Apr. 7, 2009

Related U.S. Application Data
(60) Provisional application No. 60/763,972, filed on Feb. 1, 2006.

(51) Int. Cl. A61K 9/14; A61K 31/485; A61K 31/195; A61P 25/32

(52) U.S. Cl. 424/486; 514/282; 514/567

(57) ABSTRACT

The current invention provides methods of treating alcohol related disorders by providing a sustained release oral drug dosage form comprising a plurality of solid state drugs i.e., baclofen and naltrexone, dispersed in a solid state unitary matrix formed from a combination of swellable polymers. The combination of swellable polymers in a single oral drug dosage form is beneficial in terms of release rate control for combination therapies.

### RELATED APPLICATIONS

This application is a National stage entry of International Application No. PCT/US2007/02972, which designated the United States and was filed on Feb. 1, 2007, published in English, which claims the benefit of U.S. Provisional Application No. 60/763,972, filed on Feb. 1, 2006. The entire teaching of the above application is incorporated herein by reference.

### TECHNICAL FIELD

The present invention relates to a combination therapy for the treatment of alcohol related disorders in a single once-a-day drug dosage form.

### BACKGROUND OF THE INVENTION

Alcohol dependence is a chronic disorder that results from a variety of genetic, physiological and environmental factors. Treatment has consisted of two phases: detoxification and rehabilitation. Detoxification ameliorates the symptoms and signs of withdrawal; rehabilitation helps the patient avoid future problems with alcohol. In the past, most rehabilitative treatments have been psychological. With advances in neurobiology, there is increasing interest in drug therapy for alcohol dependence. For example, studies have since examined the potential effects of various therapeutic compounds such as opioid antagonist, GABA B agonist, NMDA antagonist, serotonin antagonist, and cannabinoid antagonist as effective treatment options for alcohol dependence. Additional studies have also been conducted to examine the potential combined effect of administering two therapeutic compounds in combination therapies. For a discussion of the development of this field, see Swift, R., Drug Therapy for Alcohol Dependence, NEJM, May 13, 1999, 1482-1490. Yet, the successful treatment of alcohol dependency has many serious challenges and complications. For example, dosing regimes for combination therapies are often limited by the therapeutic compounds' absorption, distribution, metabolism, and excretion (ADME) profile, which influences the pharmacological activity of the compound as an effective drug. ADME further governs effective dosing levels of the therapeutic compound (whether a drug is administered once, twice (BID), three (TID) or four times a day (QID)). Compounds with different ADME profiles are inhibited from being combined in a single oral dosage form, thereby, patient complience and lowered dosing regimes as a result of additive or synergistic effects of the combination therapy are compromised.

As such, a further need exists for effective treatments to treat alcohol related disorders. In particular, multiple treatments to treat alcohol dependency in a single once-a-day drug dosage form.

### SUMMARY OF THE INVENTION

The current invention provides a method of treating alcohol related disorders by providing a sustained release oral drug dosage form comprising a plurality of solid state drugs dispersed in a solid state unitary matrix formed from a combination of swellable polymers. The combination of swellable polymers in a single oral drug dosage form is beneficial in terms of release rate control for combination therapies. For example, the GABA B agonist e.g., baclofen is currently administered three times daily (TID) in a dose of 40 to 80 mg, while the opioid antagonist e.g., naltrexone is administered once a day in a dose of 50 mg. The utility of the present invention provides multiple treatments for alcohol related disorders in a once-a-day drug dosage form. Such treatment has the advantage of greatly increasing patient compliance.

Furthermore, the combination treatments as described herein produce a synergistic or additive effect on an alcohol related disorder. For example, the combined effect of administering two therapeutic compounds e.g., naltrexone plus baclofen, produces an overall response that is greater than the sum of the two individual effects. The synergistic or additive effect of the combined therapy allows for a lower dosing regime than that currently available in the market place for a monotherapy.

The current invention provides a method of treating a subject suffering from an alcohol related disorder comprising administering to the stomach of a subject, a controlled release combination of an opioid antagonist and a GABA B agonist in a matrix wherein said matrix comprises a first polymeric matrix and a second polymeric matrix, and wherein the opioid antagonist is dispersed within the first polymeric matrix and the GABA B agonist is dispersed within the second polymeric matrix, said matrix being one that i) swells upon contact with water to promote retention of said oral drug dosage form in the stomach, ii) releases the opioid antagonist and the GABA B agonist into the gastric fluid by erosion of the first and second polymeric matrices by gastric fluid; and iii) exhibits different erosion rates for each of the first and second polymeric matrices.

In one aspect, the opioid antagonist of the present invention administered is selected from the group consisting of naltrexone, naloxone and nalmefene or a pharmaceutically acceptable salt, isomer, prodrug, analog, metabolite or derivative thereof.

In another aspect, the opioid antagonist of the present invention is represented by the structure of the following formula:

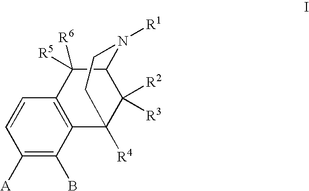

R¹ is selected from the group consisting of hydrogen, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or saturated or unsaturated heterocyclic group;

R² is selected from the group consisting of hydrogen, hydroxy, alkoxy, amino or substituted amino;

R³ and R⁴ are aliphatic;

or R³ and R⁴ are taken together with the carbon atoms to which they are attached, to form the following formula II:

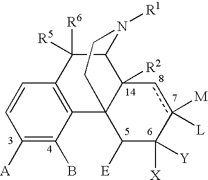

R⁵ and R⁶ are both hydrogen or taken together R⁵ and R⁶ are ═O;

A, B and E are independently selected from hydrogen, halogen, R¹, OR¹, SR¹, CONR³R⁴ and NR³R⁴; wherein R³ and R⁴ is independently selected from the group consisting of hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, saturated or unsaturated heterocyclic group; or can be taken together with the nitrogen atom to which they are attached to form a substituted or unsubstituted heterocyclic or heteroaromatic ring;

or B and E are taken together to form the following formula III:

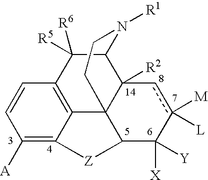

wherein Z is selected from O, S, or NR¹;

X and Y are independently selected from the group consisting of hydrogen, deuterium, halogen, nitrile, azide, R₁, OR₁, S(O)ₙR¹, —NR¹C(O)R¹, —NR¹C(O)NR³R⁴, —NR¹S(O)ₙR¹, —CONR³R⁴, and NR³R⁴;

or X and Y, taken together with the carbon atom to which they are attached, are selected from the group consisting of CO, C═CHR¹, C═NR¹, C═NOR¹, C═NO(CH₂)ₘR¹, C≡NNHR¹, C═NNHCOR¹, C═NNHCONR¹R², C═NNHS(O)ₙR¹, or C═N—N═CHR¹;

R² and either X or Y taken together to form an additional sixth ring, which may be saturated or unsaturated;

L and M are independently selected from the group consisting of hydrogen, R₁, OR₁;

or L and M, taken together with the carbon atom to which they are attached, is selected from the group consisting of C═CHR¹, or a C₃-C₁₀ spiro-fused carbocycle;

L and Y can be taken together to form a fused substituted or unsubstituted aryl or heteroaryl.

An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alkyl”, as used herein, refers to saturated, straight- or branched-chain hydrocarbon radicals containing between one or more carbon atoms. Examples of C₁-C₃ alkyl radicals include methyl, ethyl, propyl and isopropyl radicals; examples of C₁-C₆ alkyl radicals include, but are not limited to, methyl, ethyl, propyl, isopropyl, n-butyl, tert-butyl, sec-butyl, n-pentyl, neopentyl and n-hexyl radicals; and examples of C₁-C₁₂ alkyl radicals include, but are not limited to, ethyl, propyl, isopropyl, hexyl, heptyl, octyl, nonyl, decyl, undecyl, dodecyl radicals and the like.

The term “substituted alkyl,” as used herein, refers to an alkyl, such as a C₁-C₁₂ alkyl or C₁-C₆ alkyl group, substituted by one, two, three or more aliphatic substituents.

Suitable aliphatic substituents include, but are not limited to, —F, —Cl, —Br, —I, —OH, protected hydroxy, aliphatic ethers, aromatic ethers, oxo, —NO₂, —CN, —C₁-C₁₂-alkyl optionally substituted with halogen (such as perhaloalkyls), C₂-C₁₂-alkenyl optionally substituted with halogen, —C₂-C₁₂-alkynyl optionally substituted with halogen, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkynyl, —CONH—C₃-C₁₂-cycloalkyl, —CONH-aryl, —CONH-heteroaryl, —CONH-heterocycloalkyl, —CO₂—C₁-C₁₂-alkyl, —CO₂—C₂-C₁₂-alkenyl, —CO₂—C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, NHC(O)NH—C₁-C₁₂-alkyl, —NHC(O)NH—C₂-C₁₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, NHC(S)NH—C₁-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₁₂-alkyl, —NHC(NH)NH—C₂-C₁₂-alkenyl, —NHC(NH)NH—C₂-C₁₂-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, NHC(NH)—C₁-C₁₂-alkyl, —NHC(NH)—C₂-C₁₂-alkenyl, —NHC(NH)—C₂-C₁₂-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, —S-heterocycloalkyl, or methylthiomethyl. It is understood that the aryls, heteroaryls, alkyls and the like can be further substituted.

The term “alkenyl”, as used herein, denotes a monovalent group derived from a hydrocarbon moiety containing from two to twelve or two to six carbon atoms having at least one carbon-carbon double bond by the removal of a single hydrogen atom. Alkenyl groups include, but are not limited to, for example, ethenyl, propenyl, butenyl, 1-methyl-2-buten-1-yl, alkadienes and the like.

The term “alkynyl”, as used herein, denotes a monovalent group derived from a hydrocarbon moiety containing from two to twelve or two to six carbon atoms having at least one carbon-carbon triple bond by the removal of a single hydrogen atom. Representative alkynyl groups include, but are not limited to, for example, ethynyl, 1-propynyl, 1-butynyl, and the like.

The term “aryl” or “aromatic,” as used herein, refers to a mono- or bicyclic carbocyclic ring system having one or two aromatic rings including, but not limited to, phenyl, naphthyl, tetrahydronaphthyl, indanyl, idenyl and the like.

Aromatic substituents include, but are not limited to, —F, —Cl, —Br, —I, —OH, protected hydroxy, aliphatic ethers, aromatic ethers, oxo, —NO₂, —CN, —C₁-C₁₂-alkyl optionally substituted with halogen (such as perhaloalkyls), C₂-C₁₂-alkenyl optionally substituted with halogen, —C₂-C₁₂-alkynyl optionally substituted with halogen, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkynyl, —CONH—C₃-C₁₂-cycloalkyl, —CONH-aryl, —CONH-heteroaryl, —CONH-heterocycloalkyl, —CO₂—C₁-C₁₂-alkyl, —CO₂—C₂-C₁₂-alkenyl, —CO₂—C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, NHC(O)NH—C₁-C₁₂-alkyl, —NHC(O)NH—C₂-C₁₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, NHC(S)NH—C₁-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₁₂-alkyl, —NHC(NH)NH—C₂-C₁₂-alkenyl, —NHC(NH)NH—C₂-C₁₂-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, NHC(NH)—C₁-C₁₂-alkyl, —NHC(NH)—C₂-C₁₂-alkenyl, —NHC(NH)—C₂-C₁₂-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, —S-heterocycloalkyl, or methylthiomethyl. It is understood that the aryls, heteroaryls, alkyls and the like can be further substituted.

The term “arylalkyl,” as used herein, refers to an aryl group attached to the parent compound via an alkyl residue. Examples include, but are not limited to, benzyl, phenethyl and the like.

The term “heteroaryl” or “heteroaromatic,” as used herein, refers to a mono-, bi-, or tri-cyclic aromatic radical or ring having from five to ten ring atoms of which at least one ring atom is selected from S, O and N; zero, one or two ring atoms are additional heteroatoms independently selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The heteroaromatic ring may be bonded to the chemical structure through a carbon or hetero atom.

The term “cycloalkyl,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl.

The term “heterocycloalkyl,” as used herein, refers to a non-aromatic 5-, 6- or 7-membered ring or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl(trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bn or —C(O)C₆H₅), and trimethylsilyl (TMS or —Si(CH₃)₃).

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates.

In a preferred embodiment, the compound can be naltrexone or its derivatives.

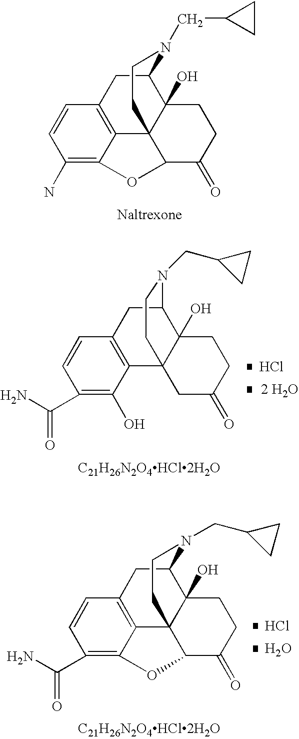

The compounds useful in the present invention are prepared using the procedures described in PCT WO02/36573 which is incorporated herein by reference.

In another aspect, the GABA B agonist is baclofen.

### DETAILED DESCRIPTION OF THE INVENTION

The present invention provides methods for treating an alcohol related disorder by providing a dosage form capable of containing separate drugs, each comprising polymers that erode at different rates. As a result, the dosage forms of the present invention achieve a plurality of drug delivery rates. Since the dosage forms of the present invention provide the drug by means of continuous, controlled release instead of conventional dosage forms, specified in terms of drug concentration and administration frequency, two particularly significant benefits result from their use: (1) a reduction in side effects from the drug through synergistic or additive effect (s); and (2) an ability to effect treatment with less frequent administration of the drug(s) being used. For instance, when administered in a conventional dosage form, naltrexone, an opioid antagonist, is administered once daily, while, baclofen, a GABA B agonist, is administered three times daily. The continued, controlled release dosage form of the present invention allows for the administration of such drugs to be combined into a once-a-day drug dosage form. Furthermore, studies have shown that the combined effect of administering two drugs e.g., naltrexone plus baclofen produces an overall response that is greater than the sum of the two individual effects. The synergistic or additive effect of the combined therapy allows for a lower dosing regime than that currently available in the market place for a monotherapy (see U.S. Ser. No. 11/591,842 incorporated herein by reference, and PCT/US2006/043221 filed Nov. 3, 2006, incorporated herein by reference).

In addition, because different drugs have different biological half-lives which determine their required frequency of administration (once, twice, three, or four times daily), thus, when two or more drugs are co-administered in one conventional medication unit, an unfavorable compromise is often required, resulting in an underdose of one drug and an overdose of the other. One of the advantages of the dosage forms of the present invention is that they can be used to deliver multiple drugs without requiring such compromises. For example, the drug/polymer composition of the present invention is designed to release the first drug i.e., naltrexone, at its ideal rate and duration (dose), while other particles contain a second drug/polymer composition designed to release the second drug i.e., baclofen, at its ideal rate and duration. In this regard, requisite erosion rates can be achieved by combining polymers of differing erosion rates into a single particle.

The term “drug” as used in the combination therapy described herein refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response. Examples of drug combinations based on the continued, controlled release dosage form of present invention include, but are not limited to, an opioid antagonist in combination with a GABA B agonist.

“Opioid antagonist” as referred to herein are compounds or compositions which serve to block the action of endogenous or exogenous opioid compounds on narcotic receptors or narcotic receptor subtypes in the brain or periphery. Opioid antagonists of the present invention are those that bind with high specificity to mu, delta or kappa receptors. Representative opioid antagonists and inverse agonists include at least one of the following: naltrexone (marketed in 50 mg dosage forms from Du Pont Pharma as ReVia™ or Trexan™), naloxone (marketed as Narcane™, NALOXONE/PENTAZOCINE™ from Pharma Pac), nalmefene, methylnaltrexone, naloxone methiodide, nalorphine, naloxonazine, nalide, nalmexone, nalbuphine, nalorphine dinicotinate, naltrindole (NTI), naltrindole isothiocyanate, (NTII), naltriben (NTB), nor-binaltorphimine (nor-BNI), b-funaltrexamine (b-FNA), BNTX, cyprodime, ICI-174,864, LY117413, MR2266, NE-100, SSR 125329, MS 377, J113397, E6276, CJ15208, LY255582 or an opioid antagonist having the same pentacyclic nucleus as nalmefene, naltrexone, buprenorphine, levorphanol, meptazinol, pentazocine, dezocine, or their pharmacologically effective esters or salts. In preferred embodiments, the opioid antagonist of the present invention is naltrexone.

A “GABA-ergic” agent is an agent that exerts a GABA-like effect, and include GABA-agonists and agents that have effects like GABA-agonists. Representative GABA agonists, antagonists and modulators include at least one of the following: muscimol, baclofen, APPA, APMPA, CaCa, valproic acid, indiplon, ocinaplon, zalepon, CGP44532, RO15-4513, RO19-4603, pregabaline, L-655,708, RY-23, AVE-1876, RU 34000, flumazenil, NGD96-3, NG2-73, CGP7930, CGP13501, GS39783, a neuroactive steroid, a barbiturate, a benzodiazepine, gabapentin, tigabine, or vigabatrin. In preferred embodiments, the GABA-ergic agonist of the present invention is baclofen.

These particular drug combinations are useful in the treatment of alcohol related disorders. As used herein, the term “patient” includes human and non-human animals such as companion animals (dogs and cats) and livestock animals. The preferred patient of treatment, amelioration and/or prevention of an alcohol related disorder is human. The terms “treating” and “treat”, as used herein, include their generally accepted meanings, i.e., preventing, prohibiting, restraining, alleviating, ameliorating, slowing, stopping, or reversing the progression or severity of an alcohol related disorder as described herein.

An alcohol related disorder of the present invention include, but are not limited to, alcohol abuse, alcohol dependence, alcohol-induced psychotic disorder, with delusions; alcohol intoxication; alcohol withdrawal; alcohol intoxication delirium; alcohol withdrawal delirium; alcohol-induced persisting dementia; alcohol-induced persisting amnestic disorder; alcohol-induced psychotic disorder, with hallucinations; alcohol-induced mood disorder; alcohol-induced anxiety disorder; alcohol-induced sexual dysfunction; alcohol-induced sleep disorder; alcohol-related disorder not otherwise specified (NOS); alcohol intoxication; and alcohol withdrawal.

The rate at which the drug is released to the gastrointestinal tract is largely dependent on the rate at which the polymer erodes. The polymer used in the continued, controlled release dosage form of the present invention should not erode and release the drug at too rapid a rate so as to provide a drug overdose or to cause the drug to pass through the gastrointestinal tract too fast (i.e., less than about four hours), nor should the polymer erode so slowly that too little of the drug is released to achieve the desired therapy. Thus, polymers having an erodibility that permits a rate of release that achieves the requisite pharmacokinetics for a desired duration are selected for use in the dosage forms of the present invention.

Polymers suitable for use in the present invention have the property of swelling as a result of imbibing water from the gastric fluid, and gradually eroding over a time period of hours. Since erosion of the polymer results from the interaction of fluid with the surface of the dosage form, erosion initiates simultaneously with the swelling process. The phrase “erosion commencing upon contact with the gastric fluid,” as used herein, refers to that erosion resulting from the contact of the gastric fluid on the surface of the dosage form exposed to that fluid. While swelling and erosion occur at the same time, the rate for achieving maximum swelling should be faster than the rate the dosage form fully erodes. More particularly, swelling should be at a rate fast enough to allow the particles to be retained in the stomach, while erosion should be of a rate that provides the desired dosing of the drug being delivered.

The water-swellable polymer forming the matrix in accordance with this invention is any polymer that is non-toxic, that swells in a dimensionally unrestricted manner upon imbibition of water, and that provides for sustained release of an incorporated drug. Examples of polymers suitable for use in this invention are cellulose polymers and their derivatives (such as for example, hydroxyethylcellulose, hydroxypropylcellulose, carboxymethylcellulose, and microcrystalline cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly(2-ethyl-2-oxazoline), poly(ethyleneimine), polyurethane hydrogels, and crosslinked polyacrylic acids and their derivatives. Further examples are copolymers of the polymers listed in the preceding sentence, including block copolymers and grafted polymers. Specific examples of copolymers are PLURONIC® and TECTONIC®, which are polyethylene oxide-polypropylene oxide block copolymers available from BASF Corporation, Chemicals Div., Wyandotte, Mich., USA.

The terms “cellulose” and “cellulosic” are used herein to denote a linear polymer of anhydroglucose. Preferred cellulosic polymers are alkyl-substituted cellulosic polymers that ultimately dissolve in the gastrointestinal (GI) tract in a predictably delayed manner. Preferred alkyl-substituted cellulose derivatives are those substituted with alkyl groups of 1 to 3 carbon atoms each. Examples are methylcellulose, hydroxymethyl-cellulose, hydroxyethylcellulose, hydroxypropylcellulose, hydroxypropylmethylcellulose, and carboxymethylcellulose. In terms of their viscosities, one class of preferred alkyl-substituted celluloses includes those whose viscosity is within the range of about 100 to about 110,000 centipoise as a 2% aqueous solution at 20° C. Another class includes those whose viscosity is within the range of about 1,000 to about 4,000 centipoise as a 1% aqueous solution at 20° C. Particularly preferred alkyl-substituted celluloses are hydroxyethylcellulose and hydroxypropylmethylcellulose. A presently preferred hydroxyethylcellulose is NATRASOL® 250HX NF (National Formulary), available from Aqualon Company, Wilmington, Del., USA.

Polyalkylene oxides of greatest utility in this invention are those having the properties described above for alkyl-substituted cellulose polymers. A particularly preferred polyalkylene oxide is poly(ethylene oxide), which term is used herein to denote a linear polymer of unsubstituted ethylene oxide. Poly(ethylene oxide) polymers having molecular weights of about 4,000,000 and higher are preferred. More preferred are those with molecular weights within the range of about 4,500,000 to about 10,000,000, and even more preferred are polymers with molecular weights within the range of about 5,000,000 to about 8,000,000. Preferred poly(ethylene oxide)s are those with a weight-average molecular weight within the range of about 1×10⁵ to about 1×10⁷, and preferably within the range of about 9×10⁵ to about 8×10⁶. Poly(ethylene oxide)s are often characterized by their viscosity in solution. For purposes of this invention, a preferred viscosity range is about 50 to about 2,000,000 centipoise for a 2% aqueous solution at 20° C. Two presently preferred poly(ethylene oxide)s are POLYOX® NF, grade WSR Coagulant, molecular weight 5 million, and grade WSR 303, molecular weight 7 million, both products of Union Carbide Chemicals and Plastics Company Inc. of Danbury, Conn., USA.

Polysaccharide gums, both natural and modified (semi-synthetic) can be used. Examples are dextran, xanthan gum, gellan gum, welan gum and rhamsan gum. Xanthan gum is preferred.

Crosslinked polyacrylic acids of greatest utility are those whose properties are the same as those described above for alkyl-substituted cellulose and polyalkylene oxide polymers. Preferred crosslinked polyacrylic acids are those with a viscosity ranging from about 4,000 to about 40,000 centipoise for a 1% aqueous solution at 25° C. Three presently preferred examples are CARBOPOL® NF grades 971P, 974P and 934P (BFGoodrich Co., Specialty Polymers and Chemicals Div., Cleveland, Ohio, USA). Further examples are polymers known as WATER LOCK®, which are starch/acrylates/acrylamide copolymers available from Grain Processing Corporation, Muscatine, Iowa, USA.

The hydrophilicity and water swellability of these polymers cause the drug-containing matrices to swell in size in the gastric cavity due to ingress of water in order to achieve a size that will be retained in the stomach when introduced during the fed mode. These qualities also cause the matrices to become slippery, which provides resistance to peristalsis and further promotes their retention in the stomach. The release rate of a drug from the matrix is primarily dependent upon the rate of water imbibition and the rate at which the drug dissolves and diffuses from the swollen polymer, which in turn is related to the solubility and dissolution rate of the drug, the drug particle size and the drug concentration in the matrix. Also, because these polymers dissolve very slowly in gastric fluid, the matrix maintains its physical integrity over at least a substantial period of time, in many cases at least 90% and preferably over 100% of the dosing period. The particles will then slowly dissolve or decompose. Complete dissolution or decomposition may not occur until 24 hours or more after the intended dosing period ceases, although in most cases, complete dissolution or decomposition will occur within 10 to 24 hours after the dosing period.

The amount of polymer relative to the drug can vary, depending on the drug release rate desired and on the polymer, its molecular weight, and excipients that may be present in the formulation. The amount of polymer will be sufficient however to retain at least about 40% of the drug within the matrix one hour after ingestion (or immersion in the gastric fluid). Preferably, the amount of polymer is such that at least 50% of the drug remains in the matrix one hour after ingestion. More preferably, at least 60%, and most preferably at least 80%, of the drug remains in the matrix one hour after ingestion. In all cases, however, the drug will be substantially all released from the matrix within about ten hours, and preferably within about eight hours, after ingestion, and the polymeric matrix will remain substantially intact until all of the drug is released. The term “substantially intact” is used herein to denote a polymeric matrix in which the polymer portion substantially retains its size and shape without deterioration due to becoming solubilized in the gastric fluid or due to breakage into fragments or small particles.

The water-swellable polymers can be used individually or in combination. Certain combinations will often provide a more controlled release of the drug than their components when used individually. Examples are cellulose-based polymers combined with gums, such as hydroxyethyl cellulose or hydroxypropyl cellulose combined with xanthan gum. Another example is poly(ethylene oxide) combined with xanthan gum.

The benefits of this invention will be achieved over a wide range of drug loadings, with the weight ratio of drug to polymer ranging in general from 0.01:99.99 to about 80:20. Preferred loadings (expressed in terms of the weight percent of drug relative to total of drug and polymer) are those within the range of 15% to 80%, more preferably within the range of 30% to 80%, and most preferably in certain cases within the range of about 30% to 70%. For certain applications, however, the benefits will be obtained with drug loadings within the range of 0.01% to 80%, and preferably 15% to 80%.

The formulations of this invention may assume the form of particles, tablets, or particles retained in capsules. A preferred formulation consists of particles consolidated into a packed mass for ingestion, even though the packed mass will separate into individual particles after ingestion. Conventional methods can be used for consolidating the particles in this manner. For example, the particles can be placed in gelatin capsules known in the art as “hard-filled” capsules and “soft-elastic” capsules. The compositions of these capsules and procedures for filling them are known among those skilled in drug formulations and manufacture. The encapsulating material should be highly soluble so that the particles are freed and rapidly dispersed in the stomach after the capsule is ingested.

In certain embodiments of this invention, the formulation contains an additional amount of the drug applied as a quickly dissolving coating on the outside of the particle or tablet. This coating is referred to as a “loading dose” and it is included for immediate release into the recipient's bloodstream upon ingestion of the formulation without first undergoing the diffusion process that the remainder of the drug in the formulation must pass before it is released. The “loading dose” is high enough to quickly raise the blood concentration of the drug but not high enough to produce the transient overdosing that is characteristic of highly soluble drugs that are not formulated in accordance with this invention.

One presently preferred dosage form is a size 0 gelatin capsule containing either two or three pellets of drug-impregnated polymer. For two-pellet capsules, the pellets are cylindrically shaped, 6.6 or 6.7 mm (or more generally, 6.5 to 7 mm) in diameter and 9.5 or 10.25 mm (or more generally, 9 to 12 mm) in length. For three-pellet capsules, the pellets are again cylindrically shaped, 6.6 mm in diameter and 7 mm in length. For a size 00 gelatin capsule with two pellets, the pellets are cylindrical, 7.5 mm in diameter and 11.25 mm in length. For a size 00 gelatin capsule with three pellets, the pellets are cylindrical, 7.5 mm in diameter and 7.5 mm in length. Another presently preferred dosage form is a single, elongated tablet, with dimensions 18 to 22 mm in length, 6.5 to 10 mm in width, and 5 to 7.5 mm in height. Still another presently preferred dosage form is a single, elongated tablet, with dimensions 18 to 22 mm in length, 6.5 to 7.8 mm in width, and 6.2 to 7.5 mm in height. A preferred set of dimensions is 20 mm in length, 6.7 mm in width, and 6.4 mm in height. These are merely examples; the shapes and sizes can be varied considerably.

The particulate drug/polymer mixture or drug-impregnated polymer matrix can be prepared by various conventional mixing, comminution and fabrication techniques readily apparent to those skilled in the chemistry of drug formulations. Examples of such techniques are as follows: (1) Direct compression, using appropriate punches and dies, such as those available from Elizabeth Carbide Die Company, Inc., McKeesport, Pa., USA; the punches and dies are fitted to a suitable rotary tableting press, such as the Elizabeth-Hata single-sided Hata Auto Press machine, with either 15, 18 or 22 stations, and available from Elizabeth-Hata International, Inc., North Huntington, Pa., USA; (2) Injection or compression molding using suitable molds fitted to a compression unit, such as those available from Cincinnati Milacron, Plastics Machinery Division, Batavia, Ohio, USA; (3) Granulation followed by compression; and (4) Extrusion in the form of a paste, into a mold or to an extrudate to be cut into lengths.

When particles are made by direct compression, the addition of lubricants may be helpful and sometimes important to promote powder flow and to prevent capping of the particle (breaking off of a portion of the particle) when the pressure is relieved. Useful lubricants are magnesium stearate (in a concentration of from 0.25% to 3% by weight, preferably less than 1% by weight, in the powder mix), and hydrogenated vegetable oil (preferably hydrogenated and refined triglycerides of stearic and palmitic acids at about 1% to 5% by weight, most preferably about 2% by weight. Additional excipients may be added to enhance powder flowability and reduce adherence.

The term “dosage form” denotes any form of the formulation that contains an amount sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular drug, including both its pharmacological characteristics and its physical characteristics such as solubility, and with the characteristics of the swellable matrix such as its permeability, and the relative amounts of the drug and polymer. In most cases, the dosage form will be such that effective results will be achieved with administration no more frequently than once every eight hours or more, preferably once every twelve hours or more, and even more preferably once every twenty-four hours or more.

As indicated above, the dosage forms of the present invention find their greatest utility when administered to a subject who is in the digestive state (also referred to as the postprandial or “fed” mode). The postprandial mode is distinguishable from the interdigestive (or “fasting”) mode by their distinct patterns of gastroduodenal motor activity, which determine the gastric retention or gastric transit time of the stomach contents.

In the interdigestive mode, the fasted stomach exhibits a cyclic activity called the interdigestive migrating motor complex (IMMC). The cyclic activity occurs in four phases: Phase I is the most quiescent, lasts 45 to 60 minutes, and develops few or no contractions. Phase II is marked by the incidence of irregular intermittent sweeping contractions that gradually increase in magnitude. Phase III, which lasts 5 to 15 minutes, is marked by the appearance of intense bursts of peristaltic waves involving both the stomach and the small bowel. Phase IV is a transition period of decreasing activity which lasts until the next cycle begins.

The total cycle time is approximately 90 minutes, and thus, powerful peristaltic waves sweep out the contents of the stomach every 90 minutes during the interdigestive mode. The IMMC may function as an intestinal housekeeper, sweeping swallowed saliva, gastric secretions, and debris to the small intestine and colon, preparing the upper tract for the next meal while preventing bacterial overgrowth. Pancreatic exocrine secretion of pancreatic peptide and motilin also cycle in synchrony with these motor patterns.

The postprandial or fed mode is induced by food ingestion, and begins with a rapid and profound change in the motor pattern of the upper GI tract, the change occurring over a period of 30 seconds to one minute. The stomach generates 3-4 continuous and regular contractions per minute, similar to those of the interdigestive mode but of about half the amplitude. The change occurs almost simultaneously at all sites of the GI tract, before the stomach contents have reached the distal small intestine. Liquids and small particles flow continuously from the stomach into the intestine. Contractions of the stomach result in a sieving process that allows liquids and small particles to pass through a partially open pylorus. Indigestible particles greater than the size of the pylorus are retropelled and retained in the stomach. Particles exceeding about 1 cm in size are thus retained in the stomach for approximately 4 to 6 hours. The dosage form of the present invention is designed to achieve the minimal size through swelling following ingestion during the fed mode.

The invention will be described in greater detail by way of specific examples. The following examples are offered for illustrative purposes, and are intended neither to limit nor define the invention in any manner.

### EXAMPLES

### Example 1

This example illustrates the use of a continued, controlled release oral drug dosage form for releasing baclofen into the stomach, duodenum and upper small intestine of a patient at a proper rate for therapeutic effectiveness following once or twice daily administration.

A controlled release oral drug dosage form as described herein has the potential to provide controlled and prolonged release of baclofen, which reduces the frequency of dosing and a potentially reduces risk of adverse side effects. The continued, controlled release oral dosing form utilizes standard-sized tablets to be retained in the stomach for 4 or more hours after administration (allowing delivery of drug at the desired rate and at the desired time), thereby extending the time of drug delivery to the GI tract. The oral tablet contains solid particles of baclofen dispersed within a polymer. Once in contact with gastric fluid in the stomach, the tablet swells to promote gastric retention. The tablet gradually erodes and releases drug to the stomach, duodenum and upper small intestine at a specific rate. This is the site of the most efficient absorption of baclofen.

In this example, the initial development approach is to determine the optimum baclofen formulation (i.e., similar plasma concentrations of baclofen as is achieved in the rat following a systemic dose combination of naltrexone and baclofen that reduces the self-administration of alcohol). Oral pharmacokinetic (PK) studies in six beagle dogs (with multiple time points from 15 minutes to 48 hours) are conducted with different formulations of baclofen incorporated within the polymer tablet to achieve a sustained plasma concentration. Plasma levels of baclofen are determined using LCMS. The plasma PK profiles for baclofen are compared.

### Example 2

This example demonstrates that naltrexone does not significantly alter the release characteristics of the controlled release-baclofen formulation (EXAMPLE 1).

In this example, a pharmacokinetic study involving the coadministration of oral naltrexone (0.5 to 100 mg/kg) as a tablet or capsule and the controlled release-baclofen tablet as is described in EXAMPLE 1. Naltrexone is readily absorbed orally and has a relatively long half-life and activity. Thus there is no need to deliver it as an extended-release oral formulation. In this example, the next phase in the development of the oral delivery of these two drugs is to conduct oral pharmacokinetic (PK) studies in six beagle dogs (with multiple time points from 15 minutes to 48 hours) with different doses of naltrexone and the optimum baclofen formulation (as described above), which is incorporated within the polymer tablet to achieve a sustained plasma concentration of baclofen. Plasma levels of baclofen and naltrexone are determined using LCMS. The PK plasma profiles of both baclofen and naltrexone for each drug combination are compared.

### Example 3

This example illustrates the use of a controlled gastric retentive form of baclofen with a rapid release coating formulation of naltrexone composing a single oral tablet in which the two drugs are released at a proper rate for therapeutic effectiveness following once or twice daily administration.

In this example, the outside layer of the controlled release-baclofen swellable polymer tablet is coated with an exact amount of naltrexone. Once swallowed, the naltrexone coating rapidly dissolves and releases naltrexone within the stomach and the controlled release polymer tablet containing baclofen would subsequently swell and gradually release baclofen over 4 or more hours. In this example, an oral PK study using beagle dogs is used to determine the PK plasma profile of both baclofen and naltrexone with each of the drug formulations tested. Plasma levels of baclofen and naltrexone are determined using LCMS.

### Example 4

This example illustrates the use of a controlled gastric retentive form of baclofen with a rapid release formulation of naltrexone composing a single oral tablet in which the two drugs are released at a proper rate for therapeutic effectiveness following once or twice daily administration.

In this example, multi-particulate dosage forms of naltrexone and baclofen are used in a controlled release swellable polymer tablet. Each drug is formulated in a different polymer (or the same polymer but with different loading factors) and combined to form a single tablet to allow the release rate and duration of release to be optimal for each drug. Once swallowed, the controlled release polymer tablet containing both naltrexone and baclofen subsequently swells, rapidly releases naltrexone while gradually releasing baclofen over 4 or more hours. In this example, an oral PK study using beagle dogs is used to determine the optimum PK plasma profile of both baclofen and naltrexone among the drug formulations tested. Plasma levels of baclofen and naltrexone are determined using LCMS.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a subject suffering from an alcohol related disorder comprising the step of orally administering to the stomach of a subject, a controlled release combination of an opioid antagonist and a GABA B agonist in a matrix wherein said matrix comprises a first polymeric matrix and a second polymeric matrix, and wherein the opioid antagonist is dispersed within the first polymeric matrix and the GABA B agonist is dispersed within the second polymeric matrix, said matrix being one that:
a) swells upon contact with water to promote retention of said oral drug dosage form in the stomach,
b) releases the opioid antagonist and the GABA B agonist into the gastric fluid by erosion of said first and second polymeric matrix by gastric fluid; and
c) exhibits different erosion rates for each of the first and second polymeric matrices.

2. The method of claim 1, wherein the opioid antagonist is selected from the group consisting of naltrexone, naloxone and nalmefene or a pharmaceutically acceptable salt, isomer, prodrug, analog, metabolite or derivative thereof.

3. The method of claim 1, wherein said opioid antagonist of the present invention is represented by the structure of the following formula: 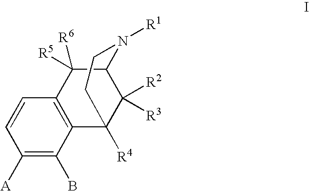
R¹ is selected from the group consisting of hydrogen, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or saturated or unsaturated heterocyclic group;
R² is selected from the group consisting of hydrogen, hydroxy, alkoxy, amino or substituted amino;
R³ and R⁴ are aliphatic;
R³ and R⁴ are taking together to form the following formula II: 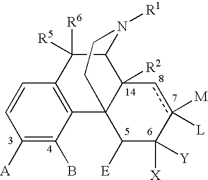
R⁵ and R⁶ are both hydrogen or taken together R⁵ and R⁶ are ═O;
A, B and E are independently selected from hydrogen, halogen, R¹, OR¹, SR¹, CONR³R⁴ and NR³R⁴; wherein R³ and R⁴ is independently selected from the group consisting of hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, saturated or unsaturated heterocyclic group; or can be taken together with the nitrogen atom to which they are attached to form a substituted or unsubstituted heterocyclic or heteroaromatic ring;
B and E are taken together to form the following formula III: 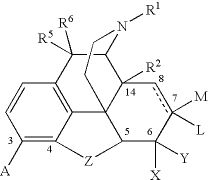
wherein Z is selected from O, S, or NR¹;
X and Y are independently selected from the group consisting of hydrogen, deuterium, halogen, nitrile, azide, R₁, OR₁, S(O)ₙR¹, —NR¹C(O)R¹, —NR¹C(O)NR³R⁴, —NR¹S(O)ₙR¹, —CONR³R⁴, and NR³R⁴;
or X and Y, taken together with the carbon atom to which they are attached, are selected from the group consisting of CO, C═CHR¹, C═NR¹, C═NOR¹, C═NO(CH₂)ₘR¹, C═NNHR¹, C═NNHCOR¹, C═NNHCONR¹R², C═NNHS(O)ₙR¹, or C═N—N═CHR¹;
R² and either X or Y taken together to form an additional sixth ring, which may be saturated or unsaturated;
L and M are independently selected from the group consisting of hydrogen, R₁, OR₁;
or L and M, taken together with the carbon atom to which they are attached, is selected from the group consisting of C═CHR¹, or a C₃-C₁₀ spiro-fused carbocycle;
L and Y can be taken together to form a fused substituted or unsubstituted aryl or heteroaryl.

4. The method of claim 1, wherein said GABA B agonist is baclofen.
